(19) United States
(12) Patent Application Publication
(10) Pub. No.: US 2022/0112235 A1
(43) Pub. Date: Apr. 14, 2022

(54) PROCESS FOR PURIFYING PHYCOCYANINS

(71) Applicant: FERMENTALG, Libourne (FR)

(72) Inventors: Olivier CAGNAC, Libourne (FR); Axel ATHANE, Carbon Blanc (FR); Julien DEMOL, Les Billaux (FR)

(21) Appl. No.: 17/420,798

(22) PCT Filed: Jan. 10, 2020
(86) PCT No.: PCT/EP2020/050547

§ 371 Date: Jul. 6, 2021

(30) Foreign Application Priority Data
Jan. 11, 2019 (FR) 1900277

(51) Int. Cl. C07K 1/14; C12N 9/26

(52) U.S. Cl. CPC C07K 1/14; C12Y 302/01015; C12N 9/2408

(57) ABSTRACT

The present invention relates to a novel process for purifying phycocyanins produced by fermenting microalgae, in particular produced by Galdieria sulphuraria, which comprises an enzymatic degradation of glycogen.

### FIELD OF THE INVENTION

The present invention relates to a novel process for purifying phycocyanins produced by fermentation of microalgae, in particular produced by Galdieria sulphuraria, which comprises enzymatic degradation of glycogen.

### PRIOR ART

The purification of phycobiliproteins extracted from Galdieria sulphuraria and Spirulina by ammonium sulfate precipitation has already been described in the literature (Moon et al., 2015; Cruz de Jesús et al., 2006), but it is very difficult to apply on an industrial scale because it requires a great deal of ammonium sulfate, which poses significant problems in reprocessing the ammonium sulfate and the supernatant.

The other purification methods described to obtain a purity level, such as chromatography methods, are very expensive to implement.

Phycocyanin extraction processes generally consist in precipitating organic matter other than phycocyanins present in an aqueous crude extract from a microalgae fermentation to preserve phycocyanins in the supernatant, which will be filtered before precipitating phycocyanins. However, some organic compounds, particularly complex polysaccharides such as glycogen, remain insensitive to this precipitation.

In an industrial phycocyanin purification process, a filtration (ultrafiltration) step can be used to remove water in order to concentrate the phycocyanin and to remove small molecules (proteins, ions, organic acid, etc.) smaller than the cut-off threshold of the filter used, in order to obtain the purest phycocyanin possible. However, the cut-off threshold of the filter being lower than the size of the glycogen, the latter is not removed and increases the viscosity of the retentate, limiting the implementation of filtration and the maintenance of its optimal parameters. The concentration-dependent viscosity effect of glycogen has been demonstrated using purified glycogen from Galdieria sulphuraria (Martinez-Garcia et al., 2017).

Moreover, the purified phycocyanins obtained retain high levels of these sugars, which may alter the properties of the purified products, in particular the coloring power, requiring a higher amount of phycocyanins for the same visual effect. These residual polysaccharides act as a filler that adds to phycocyanin manufacturing costs and may limit the commercial uses of the resulting phycocyanin, for example in the preparation of foodstuffs with a low sugar content. The presence of residual polysaccharides may limit the use of the product to the preparation of food products with a low sugar content, thus leading to an additional cost for the removal of these sugars.

Glycogen is a complex sugar that is difficult to remove if the aim is to preserve phycocyanin from the usual conditions of sugar degradation. Glycogen is a branched polyglucoside consisting of a (1-4) glucoside chains branched by a (1-6) linkages.

The use of enzymes for cell lysis is known in a process for extracting phycocyanin from a microorganism culture (CN 106749633, CN102433015 and CN1117973). This cell lysis step, breaking the cell wall to release phycocyanins, followed by extraction of the phycocyanin released in the medium, has no significant action on the glycogen released with phycocyanin and extracted with the latter.

It is possible to implement enzymatic degradation of glycogen. However, this polysaccharide is a polymer that is partially resistant to the enzymes that can degrade it. Due to the particularly large number of α1-6 glucoside branching linkages, the use of enzymes such as β-amylase (α1-4 glucosidase) is inappropriate as shown by Martinez-Garcia et al. These authors show a relatively limited activity of a pancreatic α-amylase (α1-4 glucosidase) on glycogen. The reducing sugar measurement, representing the level of digestion, remains low and saturates rapidly. The use of enzyme with α1-6 glucosidase activity (isoamylase, pullulanase) to debranch glycogen is possible as shown by the work of Martinez-Garcia et al. or Shimonaga et al. But again, the digestions are incomplete by releasing glucose polymers after long digestion times (24 to 48 hours).

These glycogen digestion experiments reported in the prior art did not integrate the problem of phycocyanin preservation even though the enzymes used can affect the integrity of phycocyanin, thus altering its coloring and antioxidant properties.

The aim is to improve processes for purifying phycocyanins extracted from biomass, both from a qualitative point of view and from an industrial and economic point of view, by reducing the residual sugar content in the final product, in particular the residual glycogen content, while preserving the phycocyanin's properties.

### DISCLOSURE OF THE INVENTION

The process in accordance with the invention consists in performing an enzymatic treatment of the phycocyanin solution to decrease the glycogen content with an enzyme suitable for degrading glycogen under temperature and pH conditions that do not substantially degrade the phycocyanins present, i.e., enzymes active at a pH below 6 and a reaction temperature below 40° C., such as glucoamylases, pectinases and pullulanases and mixtures thereof.

The process in accordance with the invention is particularly suitable for purifying acid-resistant phycobiliproteins produced by Galdieria sulphuraria, the enzymatic reaction being conducted at a pH below 6, advantageously of about 4.

The invention also relates to a phycocyanin extract with a glycogen/phycocyanin ratio (by dry weight) of less than 6, advantageously less than 4, preferably less than 3, more preferentially less than 2.5, even more preferentially less than 1.

### DESCRIPTION OF THE FIGURES

FIG. 1 represents the phycocyanin loss curves (%) over time for a pH=4 digestion at different enzyme concentrations.

FIG. 2 represents the phycocyanin loss curves (%) over time for a pH=7 digestion at different enzyme concentrations.

FIG. 3 represents the glucose release curves following glycogen digestion over time for a pH=4 digestion at different enzyme concentrations.

FIG. 4 represents the glucose release curves following glycogen digestion over time for a pH=7 digestion at different enzyme concentrations.

FIG. 5 represents change in permeate flux as a function of time for filtration of a phycocyanin extract (C-PC) with and without enzymatic digestion.

FIG. 6 represents a curve following glycogen digestion at pH=4 for different enzymes.

FIG. 7 represents a curve following glycogen digestion at pH=7 for different enzymes.

### DETAILED DESCRIPTION OF THE INVENTION

The invention relates to a process for purifying phycocyanins from a solution comprising phycocyanin(s) and glycogen, which comprises a step of enzymatically degrading the glycogen with an enzyme suitable for degrading glycogen under temperature and pH conditions that do not substantially degrade the phycocyanins present and a step of separating the phycocyanins from the glycogen degradation products.

The process in accordance with the invention is particularly suitable for purifying a phycocyanin solution extracted from a phycocyanin-producing microorganism culture which also produces glycogen, in particular in the context of an industrial phycocyanin-production process which comprises culturing the microorganisms, then recovering the biomass produced to extract the phycocyanin, and recovering the phycocyanin from this biomass.

The process is particularly suitable for phycocyanins produced by microorganisms that produce high levels of glycogen, in particular for extracting and purifying phycocyanins from biomass that comprises more than 10% glycogen based on total dry matter.

Phycocyanin-producing microorganisms are well known, in particular algae (or microalgae) of the order Cyanidiales. The order Cyanidiales includes the families Cyanidiaceae or Galdieriaceae, themselves subdivided into the genera Cyanidioschyzon, Cyanidium or Galdieria, to which belong among other species Cyanidioschyzon merolae 10D, Cyanidioschyzon merolae DBV201, Cyanidium caldarium, Cyanidium daedalum, Cyanidium maximum, Cyanidium partitum, Cyanidium rumpens, Galdieria daedala, Galdieria maxima, Galdieria partite and Galdieria sulphuraria. Particular mention may be made of the strain Galdieria sulphuraria (also known as Cyanidium caldarium) UTEX 2919.

Mention may also be made of known phycocyanin producers such as the filamentous cyanobacteria of the genus Arthrospira, which are industrially cultivated under the common name of spirulina.

The microorganisms that produce phycocyanin with a high glycogen content are particularly identified among the microorganisms mentioned above, especially species of the genera Arthrospira, Spirulina, Synechococcus, Cyanidioschyzon, Cyanidium and Galdieria, more particularly Galdieria sulphuraria.

Glycogen is a polysaccharide widely present in nature in a variety of organisms (bacteria, yeast, animal cells, etc.). If the structure of glucose polymer by α1-4 linkage branched by α1-6 linkage is common, the difference comes from the percentage and distribution of the branches. In particular, “glycogen”, in the sense of the present invention, is understood to mean the glucose polymer present in the previously mentioned phycocyanin-producing organisms, the distinctive feature of which is a majority branching size of less than 10 glucose units as illustrated by the work by Martinez-Garcia et al.

Industrial processes for culturing phycocyanin-producing microorganisms are well known to the person skilled in the art. Particular mention may be made of applications WO 2017/093345, WO 2017/050917 and WO 2018/178334.

The recovery of phycocyanin from biomass is also known to the skilled person. Particular mention may be made of application WO 2018/178334. It generally requires a step of either mechanical or enzymatic cell lysis in order to release the phycocyanin produced in the cellular compartments of the microorganisms. This cell lysis will generally generate a phycocyanin solution which comprises organic matter in suspension (called crude suspension) which can be separated by usual separation methods, in particular filtration, especially microfiltration, or centrifugation followed by filtration, more particularly by microfiltration. A crude phycocyanin solution is then obtained which can be further purified to remove low-molecular-weight organic residues by usual ultrafiltration methods to obtain a refined solution from which the phycocyanin can be obtained by usual precipitation and drying methods. Particular mention may be made of tangential filtration on ceramic membranes or organic membranes such as polyethersulfone or polysulfone hollow fibers, in particular those proposed by the company Repligen. The thresholds of these filters can be chosen to separate molecules of molecular weight higher or lower than the targeted phycocyanins.

The phycocyanin obtained can then be purified, in particular by a diafiltration step to remove low-molecular-weight organic residues as much as possible.

The enzymatic treatment in accordance with the invention can be carried out both on the crude suspension and on the crude solution.

The process in accordance with the invention is particularly suitable for purifying solutions of acid-resistant phycocyanins, in particular the phycocyanins described in application WO 2017/050918.

In particular, the process in accordance with the invention is used for purifying acid-resistant phycocyanins produced by Galdieria sulphuraria, more particularly in an industrial process for producing these phycocyanins by fermenter culture of Galdieria sulphuraria.

The preferred conditions for carrying out the enzymatic reaction are a pH below 7 and a reaction temperature below 60° C., preferably below 50° C., even more preferentially below 30° C.

Advantageously, enzymatic lysis of glycogen is carried out at a pH of less than or equal to 5, preferably of about 4.5.

Preferentially, the enzymatic reaction is carried out at room temperature. This room temperature corresponds to the definition of a use in a temperate zone or in a room with a temperature corresponding to a temperate zone, i.e., ranging from 18 to 28° C., more generally from 20° C. to 25° C.

These temperature and pH conditions are particularly suitable to preserve the phycocyanin during the enzymatic reaction.

Enzymes active under acidic pH conditions and at room temperature are known to the skilled person. However, the conditions for digesting glycogen in order to preserve phycocyanin and to facilitate its production are not known.

Surprisingly, it was found that enzymes known to have α1-4 galactosiduronic activity also have α1-4 glucosidase (or alpha-glucosidase) activity under pH and temperature conditions compatible with phycocyanin purification.

This is the case in particular for pectinases known to degrade pectin and in particular for pectinases extracted from filamentous fungi such as Aspergillus, more particularly pectinases extracted from Aspergillus aculeatus, such as the enzymes marketed under the name Pectinex® by the company Novozymes.

The action of these enzymes decreases the size of the glycosidic chains which can then be removed by ultrafiltration under conditions that allow phycocyanins to be retained while letting glycogen fragments pass.

The inventors have found that these enzymatic lysis conditions release polyglucoside chains and few glucose monomers and are therefore particularly suitable for avoiding contamination by other microorganisms, in particular organisms pathogenic to humans or animals, which is essential when the phycocyanin obtained is used as food colorant.

According to a particular embodiment, enzymatic lysis of glycogen may also be achieved with an α1-6 glucosidase activity in addition to α1-4 glucosidase or polygalacturonase activity.

The enzyme employed in the process may then be a mixture of enzymes, a first enzyme having α1-4 glucosidase activity or a polygalacturonase and a second enzyme having α1-6 glucosidase activity.

α1-6 Glucosidases active under the pH and temperature conditions set forth above are also known to the skilled person. In particular, these are pullulanases known to hydrolyze α1-6 glycosidic bonds of pullulan, in particular known to remove starch branches.

These are generally enzymes extracted from bacteria, particularly from the genus Bacillus. U.S. Pat. Nos. 6,074,854, 5,817,498 and WO 2009/075682 describe such pullulanases extracted from Bacillus deramificans or Bacillus acidopullulyticus. Commercially available pullulanases are also known, notably under the names “Promozyme D2” (Novozymes), “Novozym 26062” (Novozymes) and “Optimax L 1000” (DuPont-Genencor). It should be noted that pullulanase/alpha-amylase mixtures are described in the prior art, but in particular to produce glucose syrup from starch (US 2017/159090).

According to another preferred embodiment of the invention, the enzyme has both α1-4 glucosidase activity and α1-6 glucosidase activity.

This is the case in particular for glucoamylases. These are also enzymes extracted from microorganisms, in particular from yeasts or fungi, such as S. diastaticus or A. niger. Numerous glucoamylases are known from the prior art, described in the literature and in particular in patent applications such as WO 2019/036721. They are generally used in fermentation processes, either for the production of alcohols for consumption (beer, spirits) or for the fermentation of biomass for the production of bioethanol. They are also used as bakery additives or as food supplements. Glucoamylases are known to be commercially available, notably under the names “Amylase AG XXL” (Novozymes) or “Panzym® AG XXL” (Eaton).

Advantageously, the enzymes employed in the process in accordance with the invention are enzymes authorized for use in the food industry.

The optimal enzyme content used in this glycogen lysis step can be determined by the person skilled in the art according to the activity of the enzymes used under the temperature and pH conditions described above.

The enzyme concentrations are generally between 0.0001% and 5%, preferentially 0.0025% and 1%, more preferentially 0.005% and 0.5%, even more preferentially between 0.01% and 0.25%, the percentages being expressed as volume of enzyme solution to total volume of crude suspension or of crude suspension.

Enzyme solutions have enzyme concentrations generally ranging from 100 to 20,000 units/mL, the enzyme activity being that commonly attributed to these enzymes, as identified by the manufacturer.

The use of α1-6 glucosidases decreases the amounts of α1-4 glucosidase or polygalacturonase employed. The total enzyme concentrations (α1-4 glucuronidase+α1-6 glucosidases) are generally between 0.0001% and 5%, preferentially 0.0025% and 1%, more preferentially 0.005% and 0.5%, even more preferentially between 0.01% and 0.25%, the percentages being expressed as volume of enzyme solution to total volume of crude suspension or of crude suspension.

With α1-4 glucosidase or polygalacturonase alone or in mixture with α1-6 glucosidase, or with the enzyme with α1-4 glucosidase and α1-6 glucosidases activity, the reaction is advantageously carried out for less than 48 h, preferably less than 24 h, more preferentially from 5 h to 12 h.

For the process in accordance with the invention and more particularly for the step of isolation by tangential filtration for isolating phycocyanin, it is not necessary to obtain a total digestion of glycogen into glucose monomer. A partial digestion of the polysaccharide and its reduction to oligomers of sizes below the filtration cut-off threshold is sufficient to remove the glycogen from the suspension or from the phycocyanin solution.

The skilled person will know how to determine the appropriate time to best reduce the amount of glycogen as a function of the initial glycogen content, the amount of enzymes used and the purity sought for the phycocyanin produced.

The implementation of the reduction of glycogen by enzymatic digestion can be associated or replaced by the use of microorganisms that are able to degrade this polysaccharide. The skilled person will know how to exploit the capacities of these microorganisms to produce and secrete in the crude extract, enzymes capable of digesting glycogen, more particularly the enzymes previously mentioned. The skilled person will know how to select and exploit the capacities of these microorganisms to metabolize glycogen or the products resulting from the degradation of the polysaccharide. Advantageously, the skilled person will know how to exploit the capacities of these microorganisms to limit the growth of undesirable or pathogenic microorganisms, in particular by the synthesis of substances having antimicrobial activity.

The preferred conditions for carrying out the degradation of glycogen ex vivo or in vivo are a pH below 7 and a reaction temperature below 50° C., preferably below 40° C., even more preferentially below 37° C.

Advantageously, the degradation of glycogen ex vivo or in vivo is carried out at a pH less than or equal to 5, preferably of about 4.5 or 4.

Because of their distinctive feature of growth and degradation of polysaccharide, lactic bacteria appear particularly suitable. Among these, mention may be made of bacteria belonging to the genera Lactobacillus, Pediococcus, Tetragenococcus, Carnobacterium, Vagococcus, Leuconostoc, Weissella, Oenococcus, Atopobium, Streptococcus, Enterococcus, Lactococcus, Aerococcus, Alloiococcus, Melissococcus or Bifidobacterium.

The invention also relates to a phycocyanin extract with a glycogen/phycocyanin ratio (by dry weight) of less than 6, advantageously less than 4, preferably less than 3, more preferentially less than 2.5, even more preferentially less than 1.

According to a first embodiment, this phycocyanin extract is the crude phycocyanin suspension obtained after enzymatic lysis.

This treated crude suspension, also called “enzymatically-treated crude suspension”, comprises in particular phycocyanin released after cell lysis, glucose oligomers, products of enzymatic lysis of glycogen and residual glycogen with insolubles resulting from cell lysis in suspension.

According to a second embodiment of the invention, the phycocyanin extract is the crude phycocyanin solution obtained after separation of the crude suspension and enzymatic lysis of the glycogen, this lysis having been carried out before or after the separation of the crude suspension, or before and after separation (separation of the enzymatically-treated crude suspension and/or carrying out the enzymatic reaction on the crude solution).

This crude solution comprises in particular phycocyanin released after cell lysis, glucose oligomers, products of enzymatic lysis of glycogen and residual glycogen. This treated crude solution, also called “enzymatically-treated phycocyanin crude solution”, generally comprises from 0.1 to 10 g/L of phycocyanin, more preferentially from 1 to 5 g/L. The dry weight ratio of glycogen to phycocyanin is advantageously less than 3, preferably less than 2.5.

The enzymatically-treated crude solution in accordance with the invention may optionally be concentrated by removing a portion of the water according to the usual methods of the art carried out under conditions that substantially respect the integrity of the phycocyanin. In this case, the phycocyanin content of a concentrated enzymatically-treated crude solution will advantageously be from 10 to 50 g/L.

According to another embodiment, the phycocyanin extract is the phycocyanin isolated after extraction from the enzymatically-treated crude solution according to the methods described above.

For isolated phycocyanin, the dry weight ratio of glycogen to phycocyanin is advantageously less than 2, preferably less than 1.

According to another embodiment, the phycocyanin extract is the purified phycocyanin obtained after purification of the isolated extract according to the methods described above, in particular by diafiltration.

For purified phycocyanin, the dry weight ratio of glycogen to phycocyanin is advantageously less than 1, preferably less than 0.1.

Both isolated phycocyanin and purified phycocyanin may still contain traces of glucose oligomers, products of enzymatic lysis of glycogen.

The phycocyanin obtained has an E10 coloring power of 90 to 400, preferentially of at least 120, more preferentially of at least 150.

For an enzymatically-treated crude solution, the E10 coloring power is advantageously from 90 to 110.

For isolated phycocyanin, the E10 coloring power is advantageously from 150 to 210.

For purified phycocyanin, the coloring power is advantageously from 210 to 400.

The invention also relates to a process for producing a phycocyanin of microbial origin which comprises the steps of
(a) cultivation of phycocyanin-producing microorganisms as described above under cultivation conditions to produce a fermentation must comprising more than 30 g/L dry matter and at least 4% phycocyanin on a dry matter basis,
(b) cell lysis to release the phycocyanin produced and glycogen to obtain a crude suspension as defined above,
(c) separation of the crude suspension to recover a crude solution comprising phycocyanin and glycogen, and then optionally
(d) isolation of phycocyanin from the crude solution, then optionally
(e) purification of isolated phycocyanin,

characterized in that a step of enzymatic lysis of the glycogen is carried out with the enzymes and under the conditions defined above or a degradation by means of microorganisms, said enzymatic lysis being carried out on the crude suspension and/or on the crude solution.

Advantageously, the phycocyanin obtained is a phycocyanin that comprises less than 50% glycogen.

Cultivation methods are well known to the skilled person, in particular described in patent applications WO 2017/050917, WO 2017/093345 and WO 2018/178334.

They make it possible to obtain fermentation musts of more than 30 g/L dry matter, which can go to more than 100 g/L dry matter.

The phycocyanin content of at least 4% may reach more than 10% depending on the fermentation conditions and the cultivated strains.

The person skilled in the art will know how to determine the culture conditions according to his or her industrial phycocyanin production objective.

The separation step (c) is also known and described in the prior art, in particular by usual filtration methods, such as microfiltration, or centrifugation followed by filtration, in particular by microfiltration

The invention also relates to the use of the phycocyanins obtained as colorants, in particular as food colorants. It also relates to foods, solid or liquid, in particular beverages, which comprise a low-glycogen-content phycocyanin in accordance with the invention.

Phycocyanin used as a colorant can be in the form of enzymatically-treated crude solution, isolated phycocyanin or purified phycocyanin, as defined above.

### EXAMPLES

### Example 1—Monitoring of C-PC Concentration Before and After Enzymatic Lysis

The monitoring of the phycocyanin concentration of a crude extract is performed at pH 4 and pH 7 with different amounts of the enzyme “Pectinex”. The crude phycocyanin extract from Galdieria sulphuraria is produced according to the process described in the application WO 2018/178334. For this monitoring, the enzyme and the crude phycocyanin extract are filtered on a 0.22 μm filter. Digestion is performed at room temperature. For each kinetic point, a reading of the absorbances useful for the determination of the phycocyanin concentration are measured, in parallel with a glucose measurement after denaturation of the enzyme (95° C., 5 minutes) with the YSI 2700 Biochemical Analyzer.

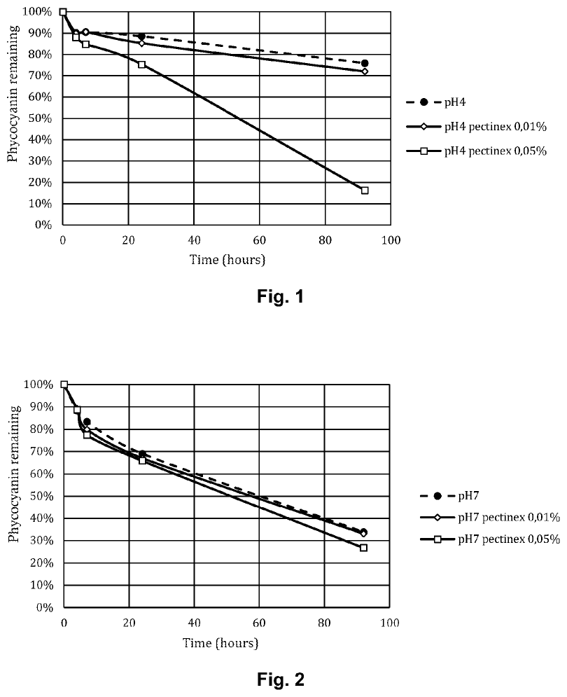

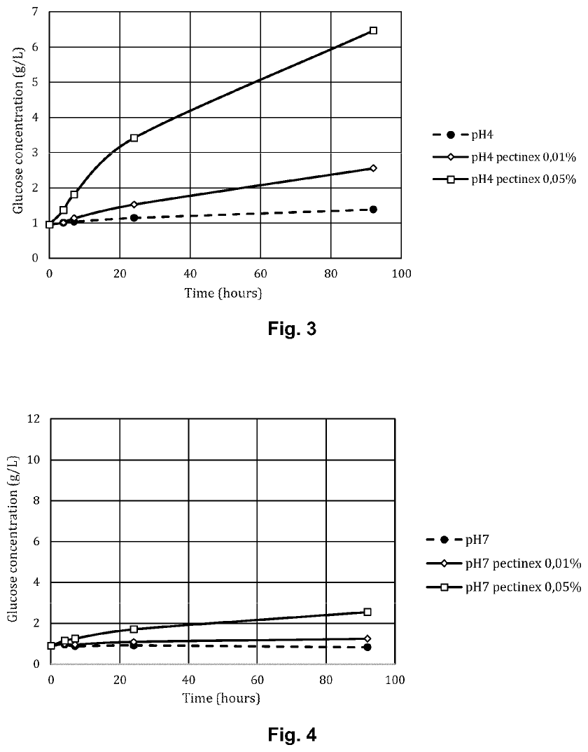

The results are shown in FIGS. 1 to 4.

These results show that the amount of digested glycogen varies between different conditions of pH and enzyme concentration. An excess of enzyme can lead to a degradation of phycocyanin.

However, we can see that after less than 24 h of digestion at pH=4 and 0.05% “Pectinex”, significant glycogen lysis is obtained while substantially limiting phycocyanin degradation.

### Example 2—Monitoring the Rate of Glycogen Digestion in a Crude Phycocyanin Solution

The monitoring of the glycogen digestion rate in a crude solution is performed at pH 4 and pH 7 with different enzymes: alpha amylase (Ban 480L from Novozymes), polygalacturonase (Pectinex Ultra SP-L from Novozymes) and glucoamylase (Amylase AG XXL from Novozymes).

The crude phycocyanin solution from Galdieria sulphuraria is produced according to the process described in the application WO 2018/178334. For this monitoring, the enzyme and the crude phycocyanin solution are filtered on a 0.22 μm filter. The digestion is performed at room temperature. For each kinetic point, a glucose measurement is performed after denaturation of the enzyme (95° C., 5 minutes) with the YSI 2700 Biochemistry Analyzer. The percentage of glycogen digestion is the ratio of the glucose concentration to the glucose concentration after total hydrolysis of the polysaccharide.

The results are shown in FIGS. 6 (pH=4) and 7 (pH=7).

### Example 3—Glycogen Content in the Purified Product with or without Enzymatic Lysis

A crude phycocyanin solution, untreated or digested 12 h with 0.25% (v/v) α1-6 glucosidases, then 2 h with 0.1% (v/v) α1-4 polygalacturonase is filtered on a hollow fiber membrane having a porosity of 70 kDa with a final diafiltration step.

The various measurements carried out at the end of each filtration and/or filtration step show that the concentration of glycogen increases significantly in the retentate until reaching non-negligible concentrations compared to the PC. It is therefore necessary to remove all or part of this glycogen to avoid diluting the coloring power of the final product, and an E10 coloring power comprised between 90 and 400.

The E10 color value (10% E618 nm) indicates the color density that is measured at 618 nm after dissolving a powder in an aqueous solution.

Protocol:

Measure 0.25 grams of the sample and dissolve it in 100 mL of citric acid buffer solution adjusted to pH 6.0. Then dilute the solution 10 times also with citric acid buffer and measure the absorbance at 618 nm using a 1 cm thick cuvette. E10 color value (10% E618 nm)=Absorbance (at 618 nm)×100/0.25 grams.

| | Glycogen (%) | C—PC in (%) | Glycogen/ | |
|---|---|---|---|---|
| Sample | of final product | of final product | PC ratio | E10 |
| Crude | 60 | 10 | 6 | 90 |
| Filtered | 50 | 20 | 2.5 | 180 |
| Digested Filtered | 20 | 28 | 0.7 | 250 |
| Digested Filtered | 3 | 40 | 0.075 | 360 |
| diafiltered | | | | |

### Example 4—Transmembrane Pressure With or Without Enzymatic Lysis

A crude phycocyanin extract from Galdieria sulphuraria produced according to the process in the patent application WO 2018/178334 is clarified on a 0.05 μm PES hollow fiber membrane. The results below present the filtration parameter monitoring with or without digestion with “Pectinex” (0.05%, 5.5 h, at room temperature and pH=4) of the same volume of 250 mL of crude extract.

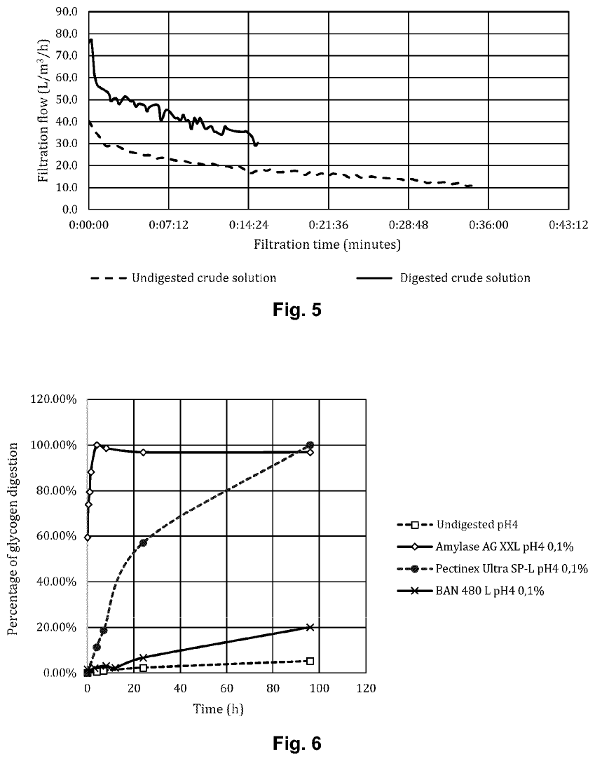

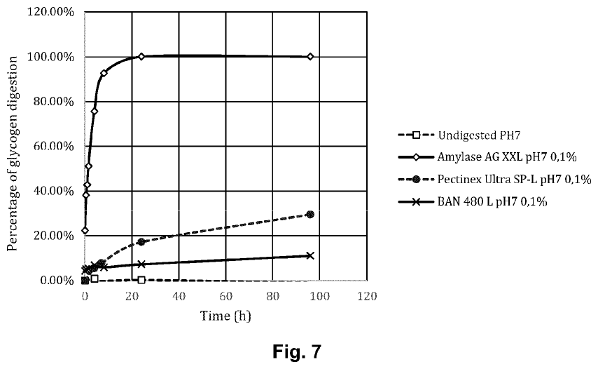

The results are shown in FIG. 5. They demonstrate the effect of glycogen digestion on the microfiltration of a crude extract. It can be seen that to filter a given volume, the time required for the digested sample is about twice as short as for the undigested sample, due to the increase in transmembrane flow.

### REFERENCES

Cruz de Jesús et al., Int J Food Nutr Sci (2016) 3(3): 1-0
Martinez-Garcia et al., Int J Biol Macromol. (2016) 89:12-8
Martinez-Garcia et al., Carbohydrate Polymers (2017) 169: 75-82
Moon et al., Korean Journal of Chemical Engineering (2014) 31, 490-495
Shimonaga et al., Marine Biotechnology (2007) 9, 192-202.
Shimonaga et al., Plant and Cell Physiology (2008) 49, 103-116.
CN 106749633, CN102433015 and CN1117973
U.S. Pat. Nos. 6,074,854, 5,817,498, US 2017/159090
WO 2009/075682, WO 2017/050917, WO 2017/050918, WO 2017/093345, WO 2018/178334, WO 2019/036721

1. A process for purifying phycocyanins from a solution comprising phycocyanin(s) and glycogen, wherein the process comprises the steps of:
(i) enzymatically degrading the glycogen with an enzyme suitable for degrading glycogen at a pH below 6 and a reaction temperature below 40° C. and
(ii) separating phycocyanins from the glycogen degradation products.

2. The process according to claim 1, wherein the temperature of step (i) is below 30° C. and/or the pH of step (i) is less than or equal to 5.

3. The process according to claim 1, wherein the enzyme has α1-4 glucosidase or polygalacturonase activity.

4. The process according to claim 3, wherein the enzyme is a pectinase.

5. The process according to claim 1, wherein the enzyme is an enzyme mixture which comprises at least one enzyme with α1-6 glucosidase activity and at least one enzyme with α1-4 glucosidase or polygalacturonase activity.

6. The process according to claim 5, wherein the enzyme with α1-6 glucosidase activity is a pullulanase.

7. The process according to claim 5, wherein the enzyme mixture comprises a pectinase and a pullulanase.

8. The process according to claim 1, wherein the enzyme has α1-4 glucosidase or polygalacturonase activity and α1-6 glucosidase activity.

9. The process according to claim 8, wherein the enzyme is a glucoamylase.

10. The process according to claim 1, wherein the solution comprising the phycocyanin(s) and glycogen is a crude suspension obtained after cell lysis of a phycocyanin-producing microorganism biomass.

11. The process according to claim 1, wherein the solution comprising the phycocyanin(s) and glycogen is a crude solution obtained after filtration of a crude suspension, the crude suspension being obtained after cell lysis of a phycocyanin-producing microorganism biomass.

12. A process for producing a phycocyanin of microbial origin which comprises the steps of:
(a) cultivation of phycocyanin-producing microorganisms as defined in claim 1 under cultivation conditions to produce a fermentation must comprising more than 30 g/L dry matter and at least 4% phycocyanin on a dry matter basis,
(b) cell lysis to release the phycocyanin produced and glycogen to obtain a crude suspension as defined above,
(c) separation of the crude suspension to recover a crude solution comprising phycocyanin and glycogen, and
(d) isolation of phycocyanin from the crude solution, and optionally:
(e) purification of isolated phycocyanin,
wherein an additional a step of enzymatic lysis of the glycogen is carried out with an enzyme suitable for degrading glycogen at a pH below 6 and a reaction temperature below 40° C., the enzymatic lysis being carried out on the crude suspension obtained in step (b) and/or on the crude solution obtained in step (c).

13. The process according to claim 12, wherein the temperature of the enzymatic lysis is below 30° C. and/or the pH of the enzymatic lysis is less than or equal to 5.

14. The process according to claim 12, wherein the enzyme has α1-4 glucosidase or polygalacturonase activity.

15. The process according to claim 14, wherein the enzyme is a pectinase.

16. The process according to claim 12, wherein the enzyme is an enzyme mixture which comprises at least one enzyme with α1-6 glucosidase activity and at least one enzyme with α1-4 glucosidase or polygalacturonase activity.

17. The process according to claim 16, wherein the enzyme with α1-6 glucosidase activity is a pullulanase.

18. The process according to claim 15, wherein the enzyme mixture comprises a pectinase and a pullulanase.

19. The process according to claim 12, wherein the enzyme has α1-4 glucosidase or polygalacturonase activity and α1-6 glucosidase activity.

20. The process according to claim 19, wherein the enzyme is a glucoamylase.

21. The process according to claim 12, wherein the solution comprising the phycocyanin(s) and glycogen is a crude suspension obtained in step (b).

22. The process according to claim 12, wherein the enzymatic lysis is carried out on the crude solution obtained in step (c).

23. The process according to claim 12, wherein the phycocyanin is a phycocyanin of microbial origin, produced by a microorganism chosen from the species of the genera Arthrospira, Spirulina, Synechococcus, Cyanidioschyzon, Cyanidium and Galdieria, more particularly Galdieria sulphuraria.

24. An isolated phycocyanin obtained by the process according to claim 1.

25. The isolated phycocyanin according to claim 24, wherein the phycocyanin it comprises traces of enzymes with alpha 1-4 and/or 1-6 glucosidase activity.

26. The isolated phycocyanin according to claim 24, wherein the phycocyanin contains glucose oligomers, products of the enzymatic lysis of glycogen.

27. A phycocyanin extract comprising phycocyanin and glycogen, wherein the dry weight ratio of glycogen to phycocyanin is less than 6, and wherein the phycocyanin extract it comprises traces of enzymes with alpha 1-4 and/or 1-6 glucosidase activity and/or glucose oligomers, products of the enzymatic lysis of glycogen.

28. The extract according to claim 27, wherein the dry weight ratio of glycogen to phycocyanin is less than 4.

29. The extract according to claim 27, wherein the dry weight ratio of glycogen to phycocyanin is less than 3.

30. The extract according to claim 27, wherein the dry weight ratio of glycogen to phycocyanin is less than 2.5.

31. The extract according to claim 27, wherein the dry weight ratio of glycogen to phycocyanin is less than 1.

32.-33. (canceled)
